(19) United States
(12) Patent Application Publication
(10) Pub. No.: US 2024/0226268 A9
(43) Pub. Date: Jul. 11, 2024

(54) TETRAVALENT DENGUE INACTIVATED VACCINE

(71) Applicant: ARMY MEDICAL UNIVERSITY, CHONGQING (CN)

(72) Inventors: Jintao Li, CHONGQING (CN); Dong Hua, CHONGQING (CN); Minchi Liu, CHONGQING (CN); Hongxia Guo, CHONGQING (CN); Minyue Qiu, CHONGQING (CN)

(21) Appl. No.: 18/330,475

(22) Filed: Jun. 7, 2023

(30) Foreign Application Priority Data
Oct. 21, 2022 (CN) 2022112959536

(51) Int. Cl. A61K 39/145; A61P 37/04

(52) U.S. Cl. CPC A61K 39/145; A61P 37/04; A61K 2039/70

(57) ABSTRACT

A tetravalent dengue inactivated vaccine is provided, including a DENV-1 inactivated antigen, a DENV-2 inactivated antigen, a DENV-3 inactivated antigen, and a DENV-4 inactivated antigen. The tetravalent dengue inactivated vaccine with a good immune effect is prepared by using four serotypes of dengue viruses as virus seeds. Furthermore, the tetravalent dengue inactivated vaccine is capable of being preserved at 4° C. for a long time, possesses lasting and effective immunogenicity, produces higher antibody titer in mice and non-human primates, has good challenge protection capability on suckling mice, has no reproductive toxicity in the mice, and has good safety.

### TECHNICAL FIELD

The disclosure relates to the field of vaccine preparation technologies, particularly to a tetravalent dengue inactivated vaccine.

### BACKGROUND

Dengue virus (DENV) belongs to a serotype subgroup of the genus Flavivirus in the family Flaviviridae, which can be divided into four serotypes of DENV-1, DENV-2, DENV-3 and DENV-4 according to their antigenicity. However, different strains in the same serotype also have antigenic differences. The dengue virus is transmitted through vector insects such as Aedes aegypti and Aedes albopictus, resulting in dengue fever (DF) and dengue hemorrhagic fever (DHF) and dengue shock syndrome (DSS) with very high morbidity and mortality. These diseases are widespread in tropical and subtropical areas, and are human infectious diseases with wide distribution, high incidence, and great hazard.

In researches on dengue vaccine, there are various types of vaccines, such as live-attenuated vaccines, recombinant subunit vaccines, inactivated purified vaccines and chimeric live-attenuated vaccines. However, these vaccines have their own disadvantages, such as virulence recovery, recombination with epidemic virus strains, immune response deviation, low success rate and low immunogenicity in the dengue virus with a high mutation rate, uneven protection efficacy of different serotypes, and limited protection effects due to infection conditions and ages before inoculation. Therefore, it is necessary to develop a dengue vaccine with a balanced immunity capacity and long-acting immunity.

### SUMMARY

An object of the disclosure is to provide a freeze-dried tetravalent dengue inactivated vaccine and a preparation method thereof, thereby make the inactivated vaccine capable of being preserved at 4 Celsius degree (° C.), and possess lasting and effective immunogenicity. Furthermore, the inactivated vaccine can produce higher antibody titer in mice and non-human primates.

In order to achieve the above object, the disclosure provides technical solutions as follows.

The disclosure provides a tetravalent dengue inactivated vaccine, including four serotypes of inactivated antigens.

The four serotypes of inactivated antigens include a dengue virus serotype 1 (DENV-1) inactivated antigen, a DENV-2 inactivated antigen, a DENV-3 inactivated antigen, and a DENV-4 inactivated antigen.

A volume ratio of the DENV-1 inactivated antigen:the DENV-2 inactivated antigen:the DENV-3 inactivated antigen:the DENV-4 inactivated antigen is 0.5-1.5:2-3:4-6:1-2.

In an embodiment of the disclosure, a volume of the four serotypes of inactivated antigens is in a range of 70% to 80% of a volume of the tetravalent dengue inactivated vaccine.

In an embodiment of the disclosure, coating quantities of the DENV-1 inactivated antigen, the DENV-2 inactivated antigen, the DENV-3 inactivated antigen, and the DENV-4 inactivated antigen each are in a range of 1.00 microgram per well (g/well) to 1.50 g/well.

In an embodiment of the disclosure, the four serotypes of inactivated antigens are prepared by:
step 1, obtaining DENV-1, DENV-2, DENV-3, and DENV-4 respectively by using Vero cells through amplification culture;
step 2, freezing and thawing the DENV-1, the DENV-2, the DENV-3, and the DENV-4 respectively for three times, and then performing a first centrifugation to obtain first supernatants respectively corresponding to the DENV-1, the DENV-2, the DENV-3, and the DENV-4, fully mixing the first supernatants with polyethylene glycol 6000 (PEG6000) respectively in a volume ratio of the first supernatant to the PEG6000 of 1:1, and then standing, performing a second centrifugation to discharge second supernatants respectively corresponding to the DENV-1, the DENV-2, the DENV-3, and the DENV-4, so as to obtain a first precipitate, a second precipitate, a third precipitate, and a fourth precipitate; and
step 3, resuspending the first precipitate obtained in the step 2 with a phosphate buffered saline (PBS) solution at a volume of 2% of a volume of the second supernatant corresponding to the first precipitate, resuspending the second precipitate obtained in the step 2 with a PBS solution at a volume of 2% of a volume of the second supernatant corresponding to the second precipitate, resuspending the third precipitate obtained in the step 2 with a PBS solution at a volume of 2% of a volume of the second supernatant corresponding to the third precipitate, resuspending the fourth precipitate obtained in the step 2 a PBS solution at a volume of 2% of a volume of the second supernatant corresponding to the fourth precipitate, and then filtering the resuspended first precipitate, the resuspended second precipitate, the resuspended third precipitate, and the resuspended fourth precipitate respectively by a filter to obtain the DENV-1 inactivated antigen, the DENV-2 inactivated antigen, the DENV-3 inactivated antigen, and the DENV-4 inactivated antigen.

In an embodiment of the disclosure, in the step 2, a time for the standing is in a range of 10 hours to 14 hours, and a temperature for the standing is in a range of 3° C. to 5° C.

In an embodiment of the disclosure, in the step 2, a temperature for the freezing and thawing is in a range of −75° C. to −85° C.

In an embodiment of the disclosure, in the step 2, a centrifugal force for the first centrifugation is in a range of 7,500 times gravity (×g) to 8,500×g, a time for the first centrifugation is in a range of 25 minutes to 35 minutes, and a temperature for the first centrifugation is in a range of 3° C. to 5° C.

In an embodiment of the disclosure, in the step 2, a centrifugal force for the second centrifugation is in a range of 9,500×g to 10,500×g, a time for the second centrifugation is in a range of 1.5 hours to 2.5 hours, and a temperature for the second centrifugation is in a range of 3° C. to 5° C.

In an embodiment of the disclosure, in the step 2, a volume concentration of the PEG6000 is in a range of 14% to 18%.

In an embodiment of the disclosure, in the step 3, a filter aperture of the filter is 0.22 micrometers (μm).

Compared with the related art, the disclosure has following beneficial effects.

The disclosure provides the freeze-dried tetravalent dengue inactivated vaccine. The tetravalent dengue inactivated vaccine is prepared by the corresponding viruses upon culture, concentration and inactivation, and then performing freezing and drying to obtain the freeze-dried tetravalent dengue inactivated vaccine. According to the disclosure, four serotypes of dengue viruses are used as virus seeds to prepare the tetravalent dengue inactivated vaccine with a good immune effect. The freeze-dried vaccine is capable of being preserved at 4° C. for a long time, possesses lasting and effective immunogenicity, produces higher antibody titer in mice and non-human primates, has a good protection of suckling mice against virus attack, has no reproductive toxicity in the mice, and has good safety.

### BRIEF DESCRIPTION OF DRAWINGS

In order to illustrate embodiments of the disclosure or the technical solutions in the related art more clearly, attached drawings required to be used in the embodiments or the related art are briefly described below. Apparently, the following attached drawings are merely used to describe the embodiments of the disclosure, and other drawings can be obtained by those skilled in the related art according to the attached drawings without paying any inventive labor.

FIGS. 1A to 1D illustrate comparison results of Immunoglobulin G (IgG) in serums of mice immunized three times with a freeze-dried vaccine preserved at 4° C. for 200 days and a ready-made vaccine.

FIG. 2 illustrates a survival curve of offspring suckling mice after intracranial challenge (with a lethal dose 80 abbreviated as LD80).

FIGS. 3A to 3D illustrate IgG levels in serums of rhesus monkeys immunized three times with the freeze-dried vaccine.

FIG. 4 illustrates detection results of immune reproductive toxicity of female mice (litter number).

FIGS. 5A to 5B illustrate detection results of immune reproductive toxicity of male mice. Specially, FIG. 5A illustrates sperm concentrations of the male mice. FIG. 5B illustrates sperm motilities of the male mice.

### DETAILED DESCRIPTION OF EMBODIMENTS

The disclosure provides a tetravalent dengue inactivated vaccine, including four serotypes of inactivated antigens.

The four serotypes of inactivated antigens include a dengue virus serotype 1 (DENV-1) inactivated antigen (DENV-1 isolate Thailand), a DENV-2 inactivated antigen (DENV-2_New Guinea C strain), a DENV-3 inactivated antigen (DENV isolate type 3), and a DENV-4 inactivated antigen (DENV-4 isolate).

A volume ratio of the DENV-1 inactivated antigen:the DENV-2 inactivated antigen:the DENV-3 inactivated antigen:the DENV-4 inactivated antigen is 0.5-1.5:2-3: 4-6:1-2. In an illustrated embodiment of the disclosure, the volume ratio is 0.7-1.3:2.5:5:1.5. In another illustrated embodiment, the volume ratio is 1:2.5:5:1.5.

In the disclosure, a volume of the four serotypes of inactivated antigens is in a range of 70% to 80% of a volume of the tetravalent dengue inactivated vaccine. In an illustrated embodiment of the disclosure, the volume of the four serotypes of inactivated antigens is in a range of 72% to 78% of the volume of the tetravalent dengue inactivated vaccine. In another illustrated embodiment, the volume of the four serotypes of inactivated antigens is in a range of 74% to 76% of the volume of the tetravalent dengue inactivated vaccine, and especially for 75%.

In the disclosure, coating quantities of the DENV-1 inactivated antigen, the DENV-2 inactivated antigen, the DENV-3 inactivated antigen, and the DENV-4 inactivated antigen each are in a range of 1.00 microgram per well (g/well) to 1.50 μg/well. In an illustrated embodiment of the disclosure, the coating quantities of the four inactivated antigens each are in a range of 1.10 μg/well to 1.40 μg/well. In another illustrated embodiment, the coating quantities of the four inactivated antigens each are in a range of 1.20 μg/well to 1.30 μg/well. In a still another illustrated embodiment, the coating quantities of the four inactivated antigens each are 1.25 μg/well.

In the disclosure, the four serotypes of inactivated antigens are prepared by:
step 1, obtaining DENV-1, DENV-2, DENV-3, and DENV-4 respectively by using Vero cells through amplification culture;
step 2, freezing and thawing the DENV-1, the DENV-2, the DENV-3, and the DENV-4 respectively for three times, and then performing a first centrifugation to obtain first supernatants corresponding to the DENV-1, the DENV-2, the DENV-3, and the DENV-4, fully mixing the first supernatants with polyethylene glycol 6000 (PEG6000) respectively in a volume ratio of the first supernatant to the PEG6000 of 1:1, and then standing, performing a second centrifugation to discharge second supernatants respectively corresponding to the DENV-1, the DENV-2, the DENV-3, and the DENV-4, so as to obtain a first precipitate, a second precipitate, a third precipitate, and a fourth precipitate; and
step 3, resuspending the first precipitate obtained in the step 2 with a phosphate buffered saline (PBS) solution at a volume of 2% of a volume of the second supernatant corresponding to the first precipitate, resuspending the second precipitate obtained in the step 2 with a PBS solution at a volume of 2% of a volume of the second supernatant corresponding to the second precipitate, resuspending the third precipitate obtained in the step 2 with a PBS solution at a volume of 2% of a volume of the second supernatant corresponding to the third precipitate, resuspending the fourth precipitate obtained in the step 2 a PBS solution at a volume of 2% of a volume of the second supernatant corresponding to the fourth precipitate, and then filtering the resuspended first precipitate, the resuspended second precipitate, the resuspended third precipitate, and the resuspended fourth precipitate respectively by a filter to obtain the DENV-1 inactivated antigen, the DENV-2 inactivated antigen, the DENV-3 inactivated antigen, and the DENV-4 inactivated antigen.

In the disclosure, in the step 2, a time for the standing is in a range of 10 hours to 14 hours, and a temperature for the standing is in a range of 3° C. to 5° C. In an illustrated embodiment of the disclosure, the time for the standing is in a range of 11 hours to 13 hours, and the temperature for the standing is at 4° C. In another illustrated embodiment, the time for the standing is 12 h, and the temperature for the standing is at 4° C.

In the disclosure, in the step 2, a temperature for the freezing and thawing is in a range of −75° C. to −85° C. In an illustrated embodiment of the disclosure, the temperature for the freezing and thawing is in a range of −77° C. to −83° C. In another illustrated embodiment, the temperature for the freezing and thawing is in a range of −79° C. to −81° C., and especially for −80° C.

In the disclosure, in the step 2, a centrifugal force for the first centrifugation is in a range of 7,500 times gravity (×g) to 8,500×g, a time for the first centrifugation is in a range of 25 minutes to 35 minutes, and a temperature for the first centrifugation is in a range of 3° C. to 5° C. In an illustrated embodiment of the disclosure, the centrifugal force for the first centrifugation is in a range of 7,700×g to 8,300×g, the time for the first centrifugation is in a range of 27 minutes to 33 minutes, and the temperature for the first centrifugation is at 4° C. In another illustrated embodiment, the centrifugal force for the first centrifugation is 8,000×g, the time for the first centrifugation is 30 minutes, and the temperature for the first centrifugation is at 4° C.

In the disclosure, in the step 2, a centrifugal force for the second centrifugation is in a range of 9,500×g to 10,500×g, a time for the second centrifugation is in a range of 1.5 hours to 2.5 hours, and a temperature for the second centrifugation is in a range of 3° C. to 5° C. In an illustrated embodiment of the disclosure, the centrifugal force for the second centrifugation is in a range of 9,700×g to 10,300×g, the time for the second centrifugation is 2 hours, and the temperature for the second centrifugation is at 4° C. In another illustrated embodiment, the centrifugal force for the second centrifugation is 10,000×g, the time for the second centrifugation is 2 hours, and the temperature for the second centrifugation is at 4° C.

In the disclosure, in the step 2, a volume concentration of the PEG6000 is in a range of 14% to 18%. In an illustrated embodiment of the disclosure, the volume concentration of the PEG6000 is in a range of 15% to 17%, and especially for 16%.

In the disclosure, in the step 3, a filter aperture of the filter is 0.22 micrometers (μm).

In the disclosure, preservation conditions for evaluating long-acting property of the vaccine are 4° C., and 200 days or more for preservation.

In the disclosure, an immune volume of a mouse is 200 microliter per dose (L/dose), and an immune volume of a rhesus monkey is 500 μL/dose.

The technical solutions provided by the disclosure are described in detail below with reference to the embodiments, but the embodiments cannot be understood as limiting the scope of the protection of the disclosure.

Vero cells used to culture the viruses of the disclosure are always preserved in the laboratory. BALB/c mice (also referred to albino inbred mice) used in the embodiments are purchased from Experimental Animal Center of Army Medical University. Other reagents or raw materials not specifically noted can be obtained by those skilled in the related art according to technologies in the related art.

### Embodiment 1

A tetravalent dengue inactivated vaccine is prepared by the following steps.

Step 1, DENV-1, DENV-2, DENV-3, and DENV-4 are obtained respectively by using Vero cells through amplification culture.

Step 2, the DENV-1, the DENV-2, the DENV-3, and the DENV-4 are frozen and thawed repeatedly for three times (at −75° C.); and then a first centrifugation (with a centrifugal force of 7,500×g, a time for 25 minutes, and a temperature at 3° C.) is performed to obtain first supernatants respectively corresponding to the DENV-1, the DENV-2, the DENV-3, and the DENV-4; the first supernatants are fully mixed with 14% of PEG6000 respectively in a volume ratio of the first supernatant to the PEG6000 of 1:1, and then are placed for standing for 10 hours at 3° C.; and a second centrifugation is performed (with a centrifugal force of 9,500×g, a time for 1.5 hours, and a temperature at 3° C.) to discharge second supernatants respectively corresponding to the DENV-1, the DENV-2, the DENV-3, and the DENV-4, so as to obtain a first precipitate, a second precipitate, a third precipitate, and a fourth precipitate.

Step 3, the first precipitate, the second precipitate, the third precipitate, and the fourth precipitate obtained in the step 2 each are resuspended with a PBS solution at a volume of 2% of a volume of the corresponding second supernatant, and then the resuspended first precipitate, the resuspended second precipitate, the resuspended third precipitate, and the resuspended fourth precipitate are respectively filtered by a 0.22 μm filter to obtain the DENV-1 inactivated antigen, the DENV-2 inactivated antigen, the DENV-3 inactivated antigen, and the DENV-4 inactivated antigen.

Step 4, the DENV-1 inactivated antigen, the DENV-2 inactivated antigen, the DENV-3 inactivated antigen, and the DENV-4 inactivated antigen are together made into a finished product of a freeze-dried vaccine.

The volume ratio of the DENV-1 inactivated antigen:the DENV-2 inactivated antigen:the DENV-3 inactivated antigen:the DENV-4 inactivated antigen is 0.5:2:4:1, the coating quantities of the DENV-1 inactivated antigen, the DENV-2 inactivated antigen, the DENV-3 inactivated antigen, and the DENV-4 inactivated antigen each are 1.00 ag/well, and the four serotypes of inactivated antigens account for 70% of the volume of the freeze-dried vaccine.

### Embodiment 2

A tetravalent dengue inactivated vaccine is prepared by the following steps.

Step 1, DENV-1, DENV-2, DENV-3, and DENV-4 are obtained respectively by using Vero cells through amplification culture.

Step 2, the DENV-1, the DENV-2, the DENV-3, and the DENV-4 are frozen and thawed repeatedly for three times (at −85° C.); and then a first centrifugation (with a centrifugal force of 8,500×g, a time for 35 minutes, and a temperature at 5° C.) is performed to obtain first supernatants respectively corresponding to the DENV-1, the DENV-2, the DENV-3, and the DENV-4; the first supernatants are fully mixed with 18% of PEG6000 respectively in a volume ratio of the first supernatant to the PEG6000 of 1:1, and then are placed for standing for 14 hours at 5° C.; and a second centrifugation is performed (with a centrifugal force of 10,500×g, a time for 2.5 hours, and a temperature at 5° C.) to discharge second supernatants respectively corresponding to the DENV-1, the DENV-2, the DENV-3, and the DENV-4, so as to obtain a first precipitate, a second precipitate, a third precipitate, and a fourth precipitate.

Step 3, the first precipitate the second precipitate, the third precipitate, and the fourth precipitate obtained in the step 2 each are resuspended with a PBS solution at a volume of 2% of a volume of the corresponding second supernatant, and then the resuspended first precipitate, the resuspended second precipitate, the resuspended third precipitate, and the resuspended fourth precipitate are respectively filtered by a 0.22 μm filter to obtain the DENV-1 inactivated antigen, the DENV-2 inactivated antigen, the DENV-3 inactivated antigen, and the DENV-4 inactivated antigen.

Step 4, the DENV-1 inactivated antigen, the DENV-2 inactivated antigen, the DENV-3 inactivated antigen, and the DENV-4 inactivated antigen are together made into a finished product of a freeze-dried vaccine.

The volume ratio of the DENV-1 inactivated antigen:the DENV-2 inactivated antigen:the DENV-3 inactivated antigen:the DENV-4 inactivated antigen is 1.5:3:6:2, the coating quantities of the DENV-1 inactivated antigen, the DENV-2 inactivated antigen, the DENV-3 inactivated antigen, and the DENV-4 inactivated antigen each are 1.50 ag/well, and the four serotypes of inactivated antigens account for 80% of the volume of the freeze-dried vaccine.

### Embodiment 3

A tetravalent dengue inactivated vaccine is prepared by the following steps.

Step 1, DENV-1, DENV-2, DENV-3, and DENV-4 are obtained respectively by using Vero cells through amplification culture.

Step 2, DENV-1, the DENV-2, the DENV-3, and the DENV-4 are frozen and thawed repeatedly for three times (at −80° C.); and then a first centrifugation (with a centrifugal force of 8,000×g, a time for 30 minutes, a temperature at 4° C.) is performed to obtain first supernatants respectively corresponding to the DENV-1, the DENV-2, the DENV-3, and the DENV-4; the first supernatants are fully mixed with 16% of PEG6000 respectively in a volume ratio of the first supernatant to the PEG6000 of 1:1, and then are placed for standing for 12 hours at 4° C.; and a second centrifugation is performed again (with a centrifugal force of 10,000×g, a time for 2 hours, a temperature at 4° C.) to discharge second supernatants respectively corresponding to the DENV-1, the DENV-2, the DENV-3, and the DENV-4, so as to obtain a first precipitate, a second precipitate, a third precipitate, and a fourth precipitate.

Step 3, the first precipitate, the second precipitate, the third precipitate, and the fourth precipitate obtained in the step 2 each are resuspended with a PBS solution at a volume of 2% of a volume of the corresponding second supernatant, and then the resuspended first precipitate, the resuspended second precipitate, the resuspended third precipitate, and the resuspended fourth precipitate are respectively filtered by a 0.22 μm filter to obtain the DENV-1 inactivated antigen, the DENV-2 inactivated antigen, the DENV-3 inactivated antigen, and the DENV-4 inactivated antigen.

Step 4, DENV-1 inactivated antigen, the DENV-2 inactivated antigen, the DENV-3 inactivated antigen, and the DENV-4 inactivated antigen are together made into a finished product of a freeze-dried vaccine.

The volume ratio of the DENV-1 inactivated antigen:the DENV-2 inactivated antigen:the DENV-3 inactivated antigen:the DENV-4 inactivated antigen is 1:2.5:5:1.5, the coating quantities of the DENV-1 inactivated antigen, the DENV-2 inactivated antigen, the DENV-3 inactivated antigen, and the DENV-4 inactivated antigen each are 1.25 ag/well, and the four serotypes of inactivated antigens account for 75% of the volume of the freeze-dried vaccine.

The following experimental embodiments use the vaccine obtained from the embodiment 3.

### Experimental Embodiment 1

Detection of Long-Acting Effectiveness of the Freeze-Dried Vaccine

5 seven-week-old BALB/c female mice are immunized by the freeze-dried vaccine (with a volume of 200 μL/dose) preserved at 4° C. for 200 days for 3 times, and an interval between the two times is 14 days. 5 seven-week-old BALB/c female mice are immunized by a ready-made vaccine (with a volume of 200 μL/dose) for 3 times, and an interval between the two times is 14 days. As for a control experiment, mice are injected with a PBS solution, and an injected volume of the PBS solution is equal to that of the vaccine.

Serums of three groups of the mice treated by the above steps are taken and are diluted according to a gradient of times of 2,000, 4,000, 8,000, 16,000, 32,000, 64,000, 128,000, and 256,000.

Four serotypes of dengue viruses (with a coating quantity of 1.25 μg/well) are respectively used as a coating antigen to perform enzyme-linked immunosorbent assay (ELISA), and then to determine serum titer of the mice. Specially, A₄₅₀ absorption (referred to an absorbance at a wavelength of 450 nanometers) illustrates an immunoglobulin G (IgG) level.

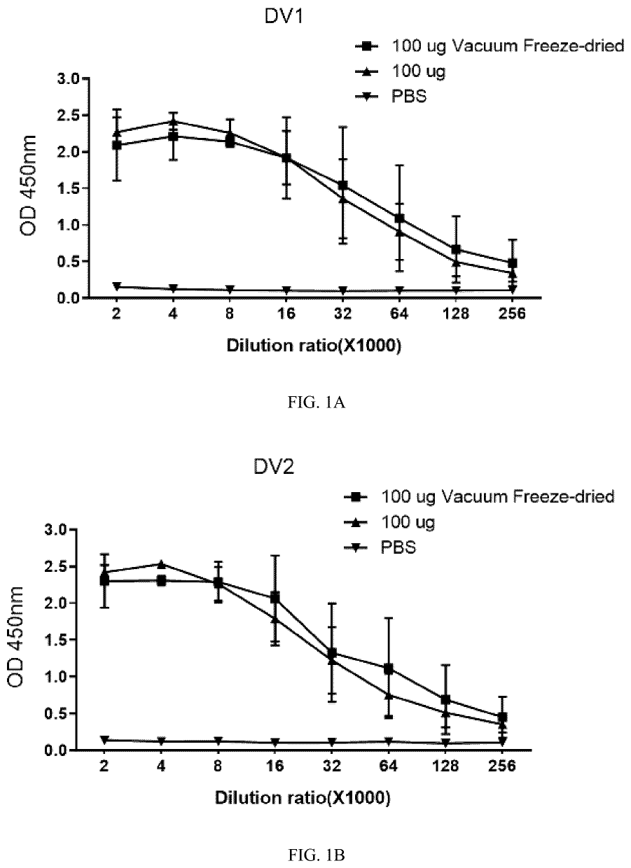

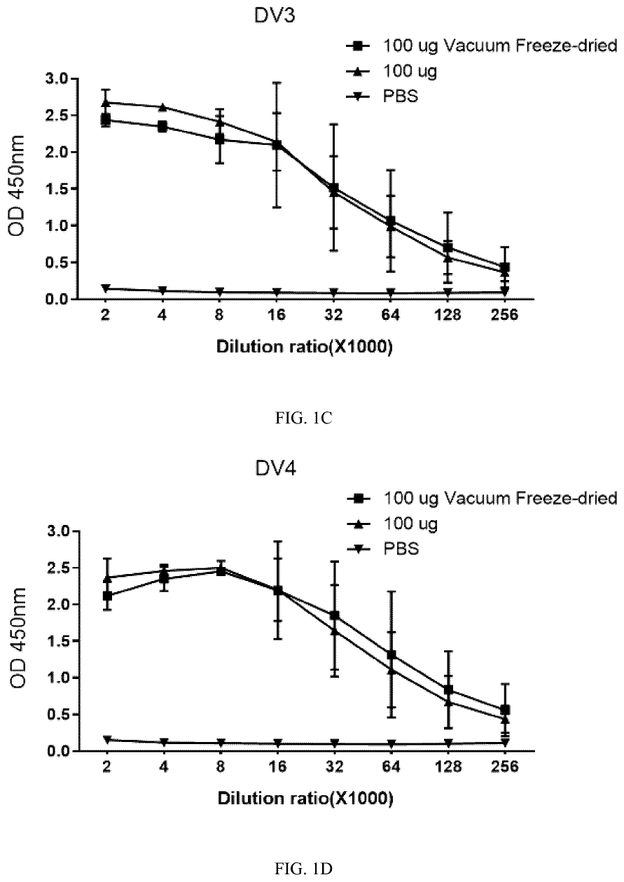

After detection, the IgG level in serums of the mice immunized by the freeze-dried vaccine preserved at 4° C. for 200 days has no significant difference from that of the mice immunized by the ready-made vaccine, which indicates that the freeze-dried tetravalent dengue inactivated vaccine performs well in long-acting effectiveness (as shown in FIGS. 1A-1D).

### Experimental Embodiment 2

Detection on Challenge Protection of Immune Mice on Offspring Suckling Mice

Seven-week-old BALB/c female mice are immunized by the freeze-dried vaccine (with a volume of 200 μL/dose) for 3 times, and an interval between the two times is 14 days. As for a control experiment, mice are injected with a PBS solution, and an injected volume of the PBS solution is equal to that of the vaccine.

After three times of immunization are completed, the mice treated by the above steps are put into a cage for pregnancy to take 3-day-old suckling mice for detection. Then, the 3-day-old suckling mice are injected with the DENV-2 in a lethal dose of 80% for intracranial challenge. Weight variations and mortalities of the suckling mice are observed daily.

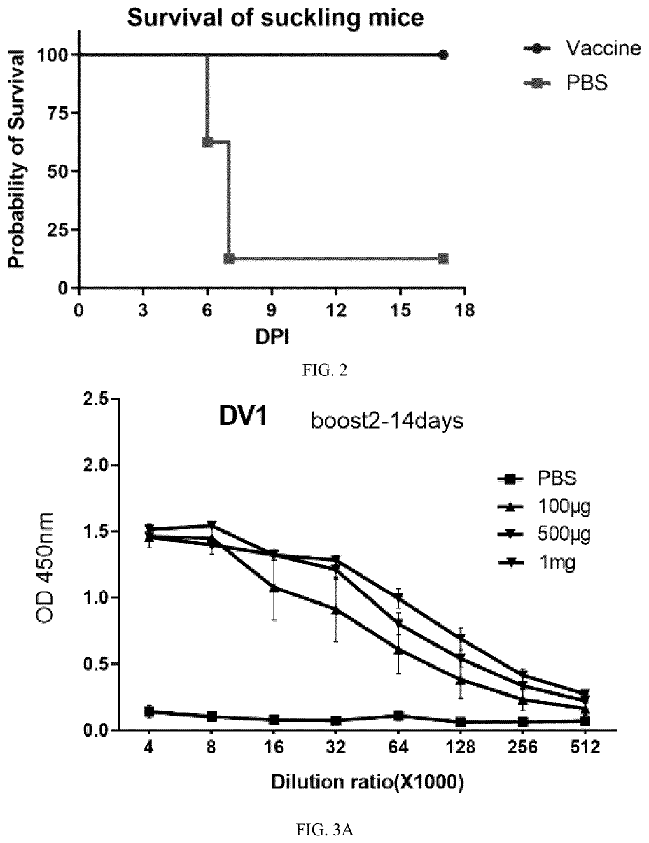

After detection, when the female mice are immunized by the tetravalent dengue inactivated vaccine, they can produce the challenge protection on the offspring suckling mice (as shown in FIG. 2).

### Experimental Embodiment 3

Detection of Immune Efficacy on Rhesus Monkeys

The rhesus monkeys are immunized by the prepared freeze-dried vaccine (with a volume of 500 μL/dose) for 3 times, and an interval between the two times is 14 days. As for a control experiment, there are four groups of rhesus monkeys, including a 100 μg dose group (one female and one male), a 500 μg dose group (two females and one male), a 1 mg dose group (two females and one male), and a PBS group (two males).

Serums of the rhesus monkeys treated after the above steps are taken and are diluted according to a gradient of times of 4,000, 8,000, 16,000, 32,000, 64,000, 128,000, 256,000, and 512,000.

Four serotypes of dengue viruses (with a coating quantity of 1.25 μg/well) are respectively used as a coating antigen to perform ELISA, and then to determine serum titer. Specially, A₄₅₀ absorption illustrates IgG level.

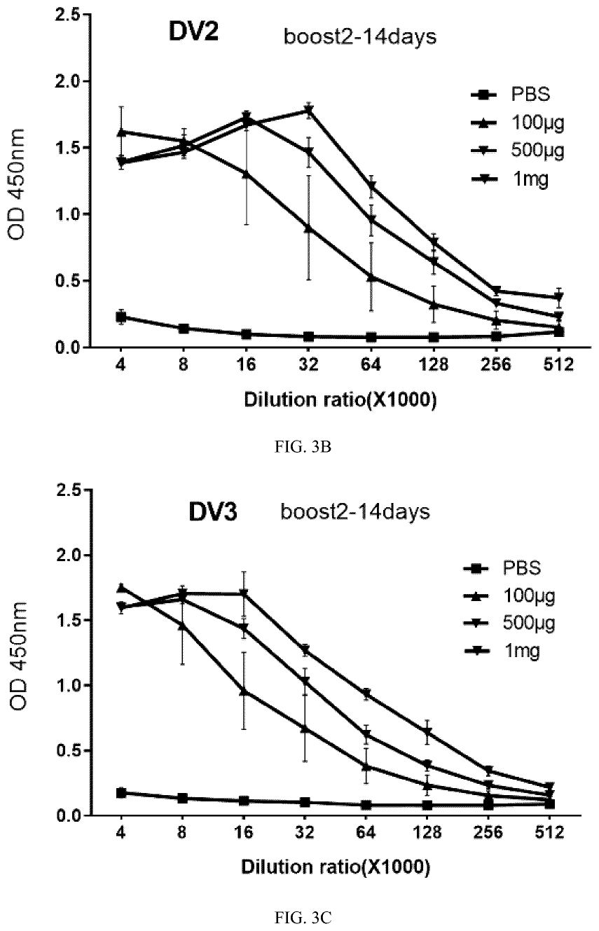

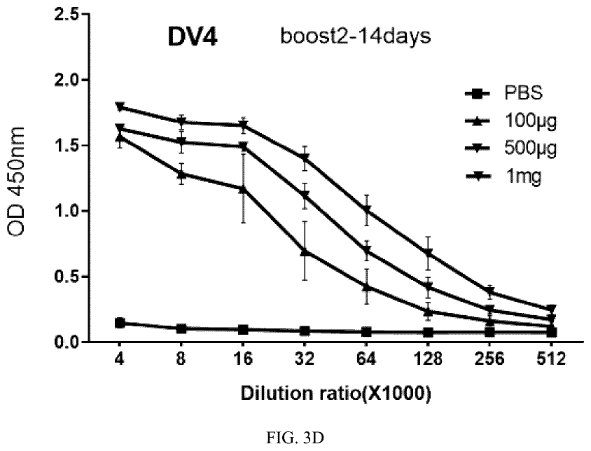

After detection, when the rhesus monkeys are immunized by the tetravalent dengue inactivated vaccine, they produce higher antibody levels. Furthermore, the antibody levels show a dose-response relationship (as shown in FIGS. 3A to 3D).

### Experimental Embodiment 4

Detection of Reproductive Toxicity in the Mice

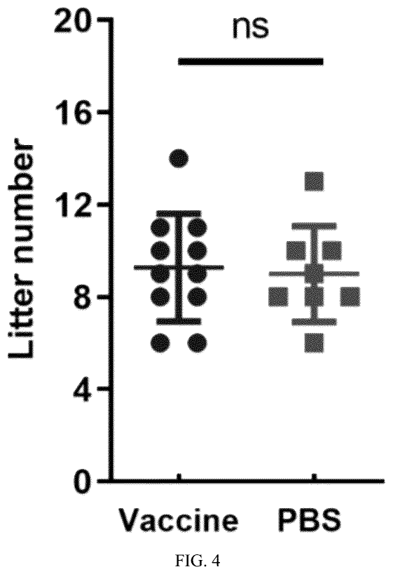

11 seven-week-old BALB/c female mice are immunized by the freeze-dried vaccine (with a volume of 200 μL/dose) preserved at 4° C. for 200 days for 3 times, and an interval between the two times is 14 days. As for a control experiment, 8 seven-week-old BALB/c female mice are injected with a PBS solution, and an injected volume of the PBS solution is equal to that of the vaccine. Thereafter, the mice are put into a cage for pregnancy to calculate farrowing rates (also referred to little number as illustrated by an ordinate of FIG. 4) of the mice treated after the above steps (as shown in FIG. 4).

There is no significant difference in the farrowing rates between the vaccine group and the PBS group, which indicates that there is no reproductive toxicity in the immunized female mice.

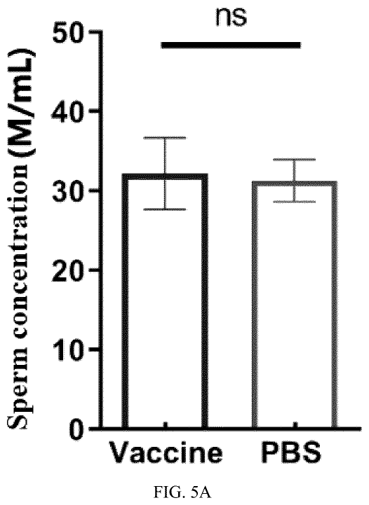

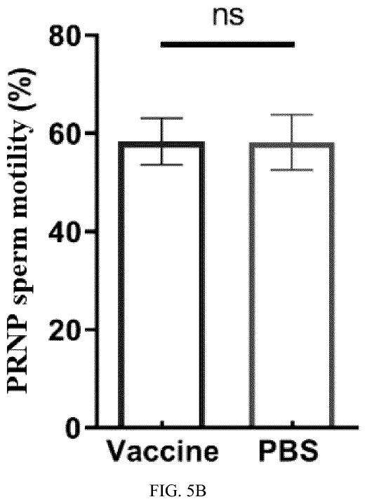

4 seven-week-old BALB/c male mice are immunized by the freeze-dried vaccine (with a volume of 200 μL/dose) preserved at 4° C. for 200 days for 3 times, and an interval between the two times is 14 days. As for a control experiment, 4 seven-week-old BALB/c male mice are injected with a PBS solution, and an injected volume of the PBS solution is equal to that of the vaccine. And then sperm concentrations (as shown in FIG. 5A) and sperm motilities (as shown in FIG. 5B) of the experimental male mice treated after the above steps are calculated.

The sperm motilities and the sperm concentrations between the vaccine group and the PBS group are not significantly different, which indicates that there is no reproductive toxicity on the immunized male mice.

The above only describes the illustrated embodiments of the disclosure. Therefore, it should be noted that, for those skilled in the related art, several improvements and modifications can be made without departing from the principle of the disclosure, and these improvements and modifications should also be regarded as the scope of the protection of the disclosure.

What is claimed is:

1. A tetravalent dengue inactivated vaccine, comprising four serotypes of inactivated antigens;
wherein the four serotypes of inactivated antigens comprise a dengue virus serotype 1 (DENV-1) inactivated antigen, a DENV-2 inactivated antigen, a DENV-3 inactivated antigen, and a DENV-4 inactivated antigen; and
wherein a volume ratio of the DENV-1 inactivated antigen:the DENV-2 inactivated antigen:the DENV-3 inactivated antigen:the DENV-4 inactivated antigen is 0.5-1.5:2-3: 4-6:1-2.

2. The tetravalent dengue inactivated vaccine according to claim 1, wherein a volume of the four serotypes of inactivated antigens is in a range of 70% to 80% of a volume of the tetravalent dengue inactivated vaccine.

3. The tetravalent dengue inactivated vaccine according to claim 1, wherein coating quantities of the DENV-1 inactivated antigen, the DENV-2 inactivated antigen, the DENV-3 inactivated antigen, and the DENV-4 inactivated antigen each are in a range of 1.00 microgram per well (ag/well) to 1.50 μg/well.

4. The tetravalent dengue inactivated vaccine according to claim 1, wherein the four serotypes of inactivated antigens are prepared by:
step 1, obtaining DENV-1, DENV-2, DENV-3, and DENV-4 respectively by using Vero cells through amplification culture;
step 2, freezing and thawing the DENV-1, the DENV-2, the DENV-3, and the DENV-4 respectively for three times, and then performing a first centrifugation to obtain first supernatants respectively corresponding to the DENV-1, the DENV-2, the DENV-3, and the DENV-4, fully mixing the first supernatants with polyethylene glycol 6000 (PEG6000) respectively in a volume ratio of the first supernatant to the PEG6000 of 1:1, and then standing, performing a second centrifugation to discharge second supernatants respectively corresponding to the DENV-1, the DENV-2, the DENV-3, and the DENV-4, so as to obtain a first precipitate, a second precipitate, a third precipitate, and a fourth precipitate; and
step 3, resuspending the first precipitate obtained in the step 2 with a phosphate buffered saline (PBS) solution at a volume of 2% of a volume of the second supernatant corresponding to the first precipitate, resuspending the second precipitate obtained in the step 2 with a PBS solution at a volume of 2% of a volume of the second supernatant corresponding to the second precipitate, resuspending the third precipitate obtained in the step 2 with a PBS solution at a volume of 2% of a volume of the second supernatant corresponding to the third precipitate, resuspending the fourth precipitate obtained in the step 2 a PBS solution at a volume of 2% of a volume of the second supernatant corresponding to the fourth precipitate, and then filtering the resuspended first precipitate, the resuspended second precipitate, the resuspended third precipitate, and the resuspended fourth precipitate respectively by a filter to obtain the DENV-1 inactivated antigen, the DENV-2 inactivated antigen, the DENV-3 inactivated antigen, and the DENV-4 inactivated antigen.

5. The tetravalent dengue inactivated vaccine according to claim 4, wherein in the step 2, a time for the standing is in a range of 10 hours to 14 hours, and a temperature for the standing is in a range of 3 Celsius degree (° C.) to 5° C.

6. The tetravalent dengue inactivated vaccine according to claim 4, wherein in the step 2, a temperature for the freezing and thawing is in a range of −75° C. to −85° C.

7. The tetravalent dengue inactivated vaccine according to claim 4, wherein in the step 2, a centrifugal force for the first centrifugation is in a range of 7,500 times gravity (×g) to 8,500×g, a time for the first centrifugation is in a range of 25 minutes to 35 minutes, and a temperature for the first centrifugation is in a range of 3° C. to 5° C.

8. The tetravalent dengue inactivated vaccine according to claim 4, wherein in the step 2, a centrifugal force for the second centrifugation is at a range of 9,500×g to 10,500×g, a time for the second centrifugation is in a range of 1.5 hours to 2.5 hours, and a temperature for the second centrifugation is in a range of 3C to 5° C.

9. The tetravalent dengue inactivated vaccine according to claim 4, wherein in the step 2, a volume concentration of the PEG6000 is in a range of 14% to 18%.

10. The tetravalent dengue inactivated vaccine according to claim 4, wherein in the step 3, a filter aperture of the filter is 0.22 micrometers (m).
